(19) United States
(12) Patent Application Publication
(10) Pub. No.: US 2011/0160147 A1
(43) Pub. Date: Jun. 30, 2011

(54) NOVEL DUAL TARGETING ANTITUMORAL CONJUGATES

(75) Inventors: Alma Dal Pozzo, Milan (IT); Emiliano Esposito, Treviglio (IT); Minghong Ni, Milan (IT); Sergio Penco, Milan (IT); Claudio Pisano, Aprilia (IT); Massimo Castorina, Rome (IT); Loredana Vesci, Rome (IT)

(73) Assignee: SIGMA-TAU INDUSTRIE FARMACEUTICHE RIUNITE S.P.A., Rome (IT)

(21) Appl. No.: 12/993,738

(22) PCT Filed: Sep. 2, 2009
(86) PCT No.: PCT/EP2009/055653

§ 371 Date: Feb. 17, 2011

(30) Foreign Application Priority Data
May 20, 2008 (EP) 08156575.6

(51) Int. Cl. A61K 38/12; C07K 7/52; A61P 35/00; A61P 35/04

(52) U.S. Cl. 514/19.5; 530/321; 514/19.9; 514/19.8

(57) ABSTRACT

The present invention relates to dual-targeting cytotoxic compounds of formula (I) and to their preparation. The described compounds are endowed with tumour specific action, incorporating three functional units: a tumour recognition moiety and a tumour selective enzymatic substrate sequence connected together by means of a spacer. These conjugates are designed to guarantee serum stability and, at the same time, the desired action inside the tumour cells as a result of enzymatic cleavability. [(L-D)ₙE]ₘ-F-D-PI-SI-CT Formula (I).

### FIELD OF THE INVENTION

The present invention relates to dual-targeting cytotoxic derivatives and their preparation. The described compounds are endowed with tumour specific action, incorporating three functional units: a tumour recognition moiety and a tumour selective enzymatic substrate sequence. These conjugates are designed to guarantee serum stability and, at the same time, the desired action inside the tumour cells as a result of enzymatic cleavability.

### BACKGROUND OF THE INVENTION

Traditional cancer chemotherapy is based on the assumption that rapidly proliferating cancer cells are more likely killed than quiescent cells of physiological tissues. Actually, cytotoxic agents have very poor specificity, causing severe undesirable effects. In the last three decades, various systems have been explored to selectively deliver drugs at their site of action. Recent improvements in the knowledge of typical receptors over expressed by cancer cells during their proliferation allow the exploitation of selective ligands, which, conjugated with cytotoxic agents, are able to preferentially address them to the tumours. Unlike the common pro-drugs, the linker between the ligand and the drug must be stable in the circulation and, following internalization of the whole conjugate into the cancer cell, should be readily cleaved, by chemical or enzymatic mechanism, to regenerate the cytotoxic agent.

Recent advances in tumour-targeting drug conjugates entail monoclonal antibodies, polyunsaturated fatty acids, hyaluronic acid and oligopeptides as ligands of tumour-associated receptors.

At present, several immunoconjugates are in clinical trials: Maytansin (Liu C., et al., Proc. Natl. Acad. Sci., 1996, 93, 8618), doxorubicin (Saleh M. N., et al., J. Clin. Oncol., 2000, 18, 11, 2282), herceptin (Baselga J., et al., J. Clin. Oncol., 1996, 14, 737), calicheamicin (Bross P. F., et al., Clin. Cancer Res., 2001, 7, 1490; Chan S. Y., et al., Cancer Immunol. Immunother., 2003, 52, 243). Regarding the latter, Mylotarg, a CD33 antibody-linked calicheamicin, was approved by FDA in 2000 for the treatment of acute leukaemia (Hammann P. R., et al., Bioconjugate Chem., 2002, 13, 1, 47).

The practical use of immunoconjugates is only suitable for highly potent drugs, because a limited amount of antigens are over expressed on tumour cell surface and only a limited number of molecules can be loaded on each mAb without decreasing the binding affinity and increasing the immunogenicity. Recently, a number of conjugates of cytotoxic agents with oligopeptides addressed to different receptors over expressed by tumoural cells have been studied as potential selective antitumoural chemotherapeutics. Among oligopeptides, the most promising seem to be somatostatin (Pollak M. N., et al., Proc. Soc. Exp. Biol. Med., 1998, 217, 143; Fuselier J. A., et al., Bioorg. Med. Chem. Lett., 2003, 13, 799), bombesin (Moody T. W., et al., J. Biol. Chem., 2004, 279, 23580), integrins-mediated RGD peptides (WO200117563, Ruoslahti E., Nature reviews Cancer, 2002, 2, 83; Dickerson E. B., et al., Mol. Cancer. Res., 2004, 2, 12, 663; de Groot F. M., et al., Mol. Cancer. Ther., 2002, 1, 901; Chen X., et al., J. Med. Chem., 2005, 48, 1098). Generally experimented chemical linkers between the tumour-recognition moiety and the anticancer drug involves hydrazones, disulfides and peptides substrates of lysosomial enzymes. The nature of the linker is the prerequisite to determine the fate of the conjugate in vivo, its stability, solubility and bioavailability.

The tumour-targeting conjugates of the present invention are made of three functional units (a tumour recognition moiety and an anticancer drug) connected together by means of a spacer (linker).

WO05111064, in the name of the Applicant, describes cyclopeptides presenting the RGD unit, endowed with anti-integrin activity. WO05111063, in the name of the Applicant, reports 7-imino camptothecin derivatives conjugated to integrin-recognizing cyclic peptides via a spacer.

WO05110487, in the name of the Applicant, reports camptothecin derivatives conjugated in position 20 to integrin antagonist.

### DESCRIPTION OF THE INVENTION

The object of the present invention is the development of tumour-targeting conjugates containing an integrin αᵥ β₃ and αᵥ β₅ recognition moiety connected to a cytotoxic drug by new molecular bridges containing three units. The latter are made of a spacer, a peptide cleavable by tumour-associated enzymes and a self-immolative functional unit.

The selected spacers are made of small flexible glycols alternate with hydrophilic amino acids or heterocyclic structures functioning as rigid moieties, that confer solubility to the whole conjugate, without interfering with the binding to the receptor. These particular spacers are superior to the widely used high molecular weight glycols, which possess great solubilizing properties, but are not advisable for their tendency to form loops that disturb the binding area.

A number of linker-containing peptides as substrates of Cathepsin B have already been described, for example, Phe-Lys, Val-Cit (Dubowchick G. M., et al, Bioconjugate Chem., 2002, 13, 4, 855); Gly-Phe-Leu-Gly (Rejmanova P., et al, Biomaterials, 1985, 6, 1, 45); D-Ala-Phe-Lys (de Groot F. M. H., et al., Mol. Cancer. Ther., 2002, 1, 901). Some of these peptides have been successfully applied when attached to antibodies, which, due to their bulkiness, can shield them from plasma peptidases. However, when we experimented these peptide sequences applied to conjugates containing small ligands, as in the case of oligopeptides, they were immediately cleaved releasing the cytotoxic agent into the circulation, contrary to what described by other authors. In particular, the Phe-Lys linker containing peptide (ST3280) resulted highly unstable in various assays conducted. The above cited paper from Dubowchick deals with cathepsin B-labile dipeptide ligands. The same authors also published four years before another study about the influence of the amino acid at position P₂ when Cit amino acid was at P₁ position, concluding that the best amino acid in such a position was Val because of hydrophobic interactions within the binding site of cathepsin B (Dubowchick G. M., et al, Bioorg. Med. Chem., 1998, 8, 3341), meanwhile the analogue containing Ala instead of Val contributed to slower noticeably the release of doxorubicin, which was clearly contrary to the objective of the study.

Surprisingly, it has now been found that Ala-Cit or D-Ala-Cit, which, unexpectedly, showed to be stable in the murine blood and cleavable inside the tumour cell are particularly well suited as a mean for allowing the release of the cytotoxic motif at the site of action.

The presence of a self-immolative group is also compulsory for exalting the endopeptidases action (Carl P. L., et al J. Med. Chem., 1981, 24, 5, 479; Shamis M. L., et al., J. Am. Chem. Soc., 2004, 126, 6, 1726). These new linkers better guarantee the required pharmacological properties of the relative conjugates, such as metabolic stability and further release of the cytotoxic agent after internalization within the cell, together with an optimal solubility and bioavailability. Furthermore, they have been designed in order to have size and conformation compatible with the binding of the targeting device to the receptor.

The new linkers are versatile molecular bridges that can be applied to a variety of ligands as well as to different antitumoural drugs.

The invention comprises compounds of general formula I

[(L-D)ₙE]ₘ-F-D-PI-SI-CT Formula I

wherein,
L is a recognizing α-integrin receptor cyclic peptide of formula II

c(R¹-Arg-Gly-Asp-R²) Formula II

### R¹ is Amp, Lys or Aad;

R² is Phe, Tyr or Amp with the R-configuration;
D at each occurrence can be the same or different, is absent or is a divalent group of formula III

-SP¹-A¹-SP²-A²-SP³- Formula III

SP¹ is absent or is R³—(CH₂)_{q}—(OCH₂—CH₂)_{q}—O—(CH₂)_{q}—R⁴;
R³ and R⁴, the same or different, are absent, or —CO—, —COO—, —NH—, —O—, or a divalent radical of formula IV, formula VIII or formula IX

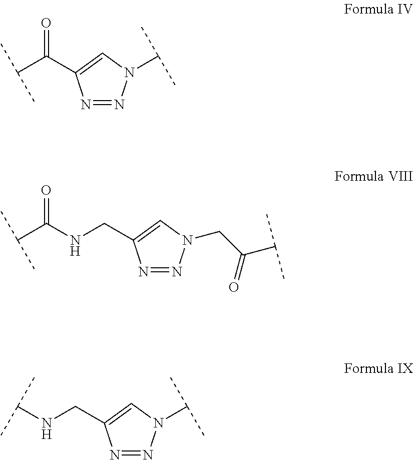

q at each occurrence can be the same or different and are independently an integer comprised between 0-6;
A¹ is absent or a natural or unnatural, (L) or (D)-amino acid bearing a hydrophilic side chain;
SP² is absent or the same as SP¹;
A² is absent or the same as A₁;
SP³ is absent or the same as SP¹;
m=1 or 2;
n=1 or 2;
E at each occurrence can be the same or different and is Glu, Lys or is absent;
F is the same as E or is absent or is a histidine analogue of formula X;

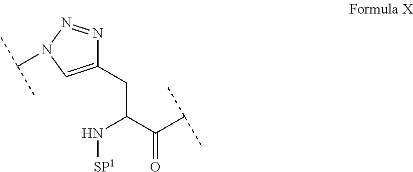

wherein the triazole ring is linked to the D-PI-SI-CT moiety, the carbonyl moiety is linked to the L-containing moiety and SP¹ is as defined above; PI is a natural or unnatural oligopeptide, made of (L) or (D) amino acids selected between Ala and Cit;
SI is the divalent radical p-aminobenzyloxycarbonyl;
CT represents a cytotoxic radical;
their tautomers, their geometrical isomers, their optically active forms such as enantiomers, diastereomers and their racemate forms, as well as their pharmaceutically acceptable salts thereof;
with the following proviso:
at least one D should be present;
and when E is present, it is linked to the portion bearing the L group through its amino moieties when E is Lys, or through its carboxyl moieties when E is Glu.

An embodiment of this invention is that of compounds of formula I, wherein CT represents a camptothecin derivative.

Another embodiment of this invention is that of compounds of formula I, wherein CT represents a camptothecin derivative, R¹ is Amp and R² is Phe.

A further embodiment of this invention is that of compounds of formula I, wherein PI represents an oligopeptide comprising two or three amino acids residues.

An even further embodiment of this invention is that of compounds of formula I, wherein m=1 and n=1.

Another preferred embodiment of this invention is that of compounds of formula I, wherein m=1 and n=2.

Compounds of formula I, can be obtained using standard coupling method well known to those skilled in the art. It will be appreciated that where typical or preferred experimental conditions (i.e. reaction temperatures, time, moles of reagents, solvents, etc.) are given, other experimental conditions can also be used, unless otherwise stated. Optimum reaction conditions may vary with the particular reactants or solvents used, but such conditions can be determined by one skilled in the art by routine optimisation procedures.

The invention furthermore provides a process for the preparation of compounds of general formula (I) for example by reacting the free amino group of the PI fragment of a compound of formula V

(CT-SI-PI)-NH₂ (Formula V)

wherein CT, SI and PI are as described above,
with an azide containing derivative of formula VI

L-(SP¹-A¹-SP²-A²-SP³)-N₃ (Formula VI)

wherein L, SP¹, A¹, SP², A² and SP³ are as described above with R⁴ being CO Alternatively, compounds of formula I can be obtained by reacting a compound of formula VII

(CT-SI-PI)-CO—C≡CH (Formula VII)

wherein CT, SI and PI are as described above,
with compounds of formula VI,
wherein L, SP¹, A¹, SP², A² and SP³ in the compounds of formula VI are as described above with the proviso that R⁴ is absent, as described by Rostovtsev V. V., et al, Angew. Chem., 2002, 41, 2596.

Compounds of formula I can also be obtained by reacting a compound of formula XI

(CT-SI-PI)-D-NHCH₂—C≡CH (Formula XI)

wherein CT, SI, PI and D are as described above,
with compounds of formula XII

[(L-D)ₙE]ₘ-COCH₂—N₃ (Formula XII)

wherein L, D and E are as described above.

Alternatively, compounds of formula I can be obtained by reacting a compound of formula XIII

(CT-SI-PI)-D-N₃ (Formula XIII)

wherein CT, SI, PI and D are as described above,
with compounds of formula XIV

[(L-D)ₙE]ₘ-CO—CH(NHD)CH₂—C≡CH (Formula XIV)

wherein L, D and E are as described above.

Amino acids bearing a hydrophilic side chain refer to amino acids chosen from the group consisting of arginine, asparagine, aspartic acid, citrulline, cysteine, glutamic acid, glutamine, histidine, lysine, serine, threonine and tyrosine.

A camptothecin derivative or cytotoxic radical means a camptothecin such as the derivatives described in WO00/53607 and WO04/083214 filed in the name of the Applicant.

Another object of the present invention is a method of treating a mammal suffering from an uncontrolled cellular growth, invasion and/or metastasis condition, comprising administering a therapeutically effective amount of a compound of Formula (I) as described above. The term “therapeutically effective amount” as used herein refers to an amount of a therapeutic agent needed to treat, ameliorate a targeted disease or condition, or to exhibit a detectable therapeutic effect.

For any compound, the therapeutically effective dose can be estimated initially either in cell culture assays or in animal models, usually mice, rats, guinea pigs, rabbits, dogs, or pigs. The animal model may also be used to determine the appropriate concentration range and route of administration. Such information can then be used to determine useful doses and routes for administration in humans. In calculating the Human Equivalent Dose (HED) it is recommended to use the conversion table provided in Guidance for Industry and Reviewers document (2002, U.S. Food and Drug Administration, Rockville, Md., USA).

The precise effective dose for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.01 mg/kg to 100 mg/kg, preferably 0.05 mg/kg to 50 mg/kg. Compositions may be administered individually to a patient or may be administered in combination with other agents, drugs or hormones.

The medicament may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity.

Suitable carriers may be large, slowly metabolised macromolecules such as proteins, polysaccharides, polylactic acids, polyglycolic acids, polymeric amino acids, amino acid copolymers and inactive virus particles.

A thorough discussion of pharmaceutically acceptable carriers is available in Remington's Pharmaceutical Sciences (Mack Pub. Co., N.J. 1991). Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The medicament of this invention may be administered by any number of routes including, but not limited to, oral, intravenous, intramuscular, intra-arterial, intramedullary, intrathecal, intraventricular, transdermal or transcutaneous applications, subcutaneous, intraperitoneal, intranasal, enteral, topical, sublingual, intravaginal, rectal means or locally on the diseased tissue after surgical operation.

Dosage treatment may be a single dose schedule or a multiple dose schedule. A further object of the present invention is a pharmaceutical composition containing at least one formula (I) compound as an active ingredient, in an amount such as to produce a significant therapeutic effect. The compositions covered by the present invention are entirely conventional and are obtained using methods that are common practice in the pharmaceutical industry. According to the administration route opted for, the compositions will be in solid or liquid form and suitable for oral, parenteral or intravenous administration. The compositions according to the present invention contain, along with the active ingredient, at least one pharmaceutically acceptable vehicle or excipient.

### DESCRIPTION OF THE DRAWINGS

FIG. 1: Describes the chemical structures of the various fragments used to synthesize dual-targeting cytotoxic derivatives.

FIG. 2: Describes the chemical structures of dual-targeting cytotoxic derivatives.

FIG. 3: Describes the synthesis of some building blocks used for the synthesis of Fragments 1, 2, 5, 6 and 12 as well as the full synthesis of Fragment 10 (FIG. 3.e).

FIG. 4: Describes schematically the nature of the two fragments required to synthesize each final compounds.

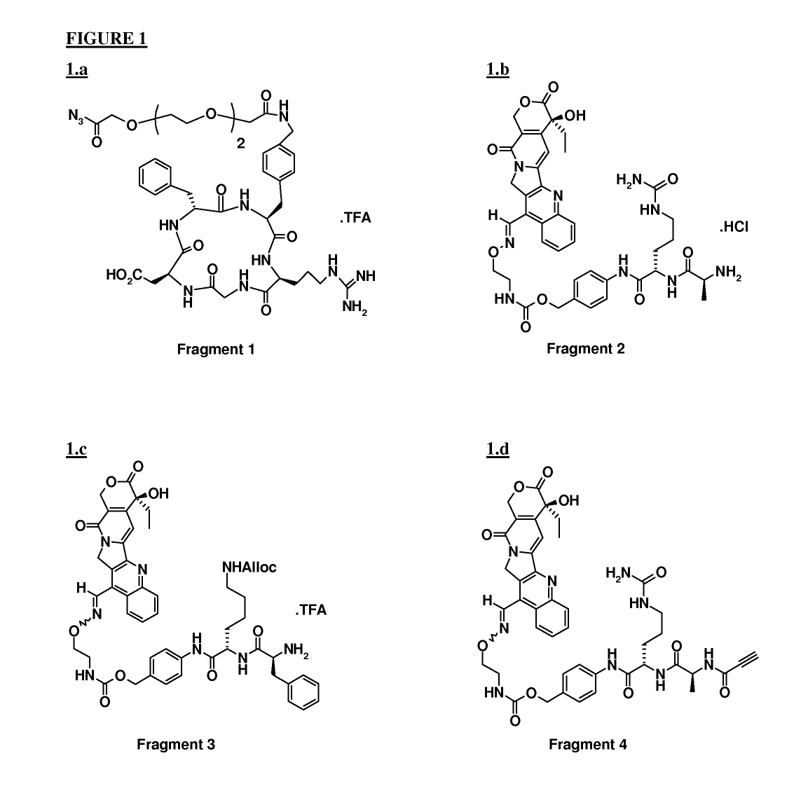

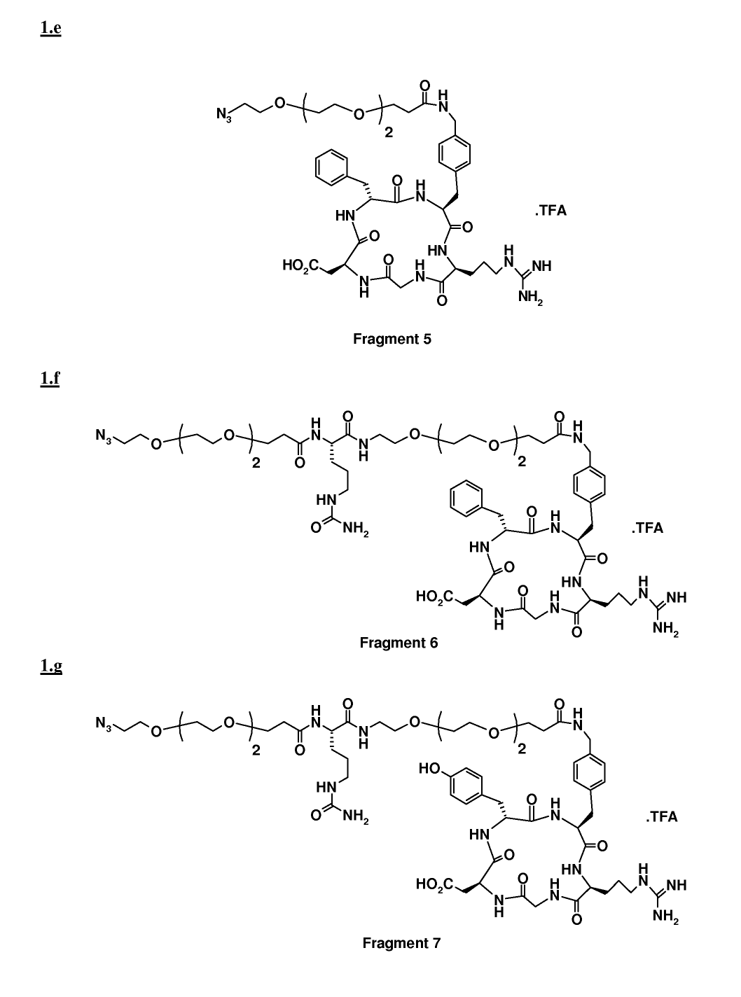

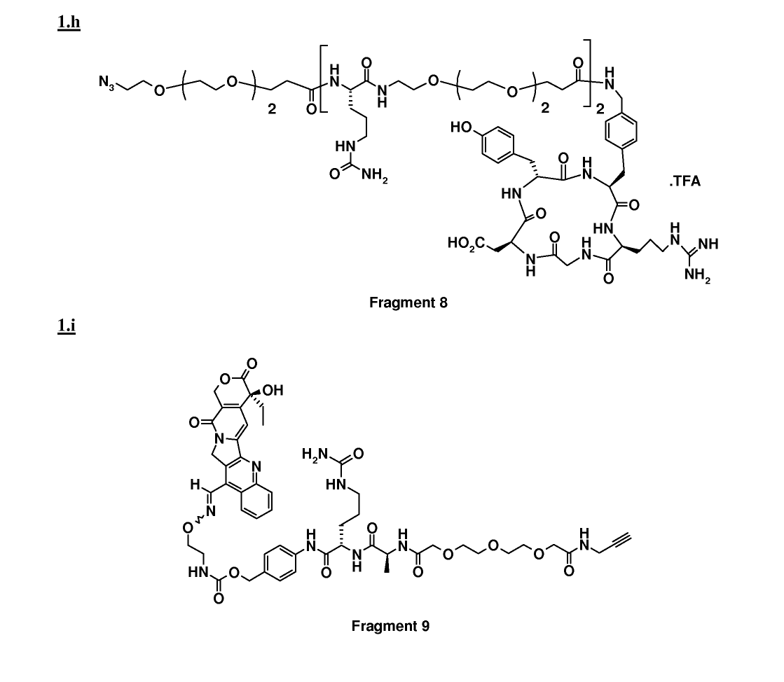

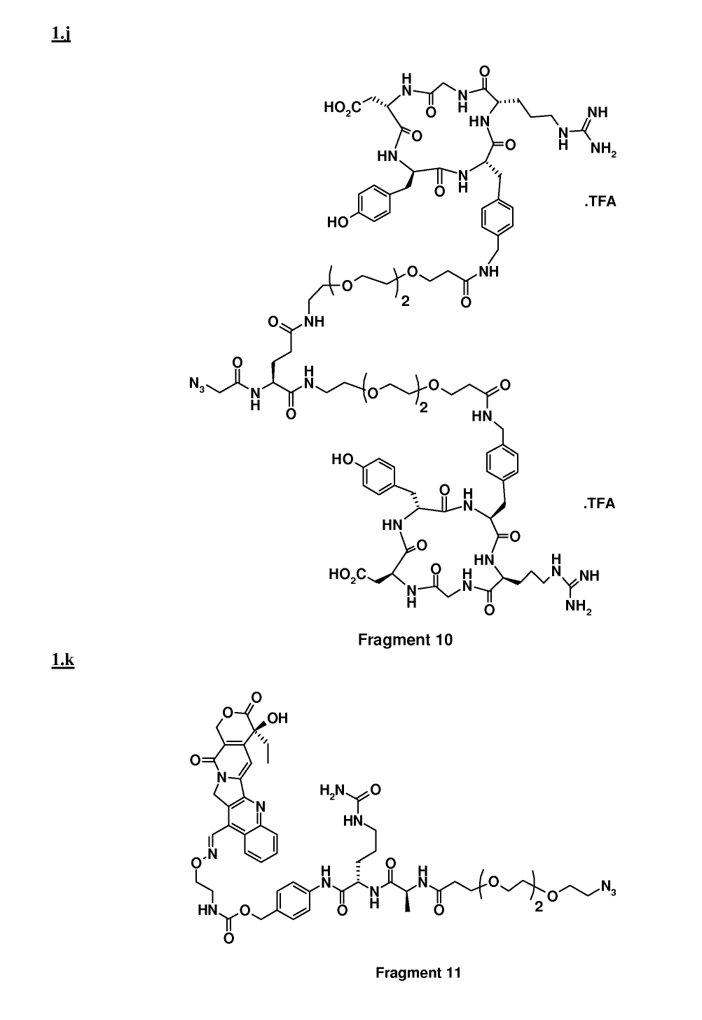

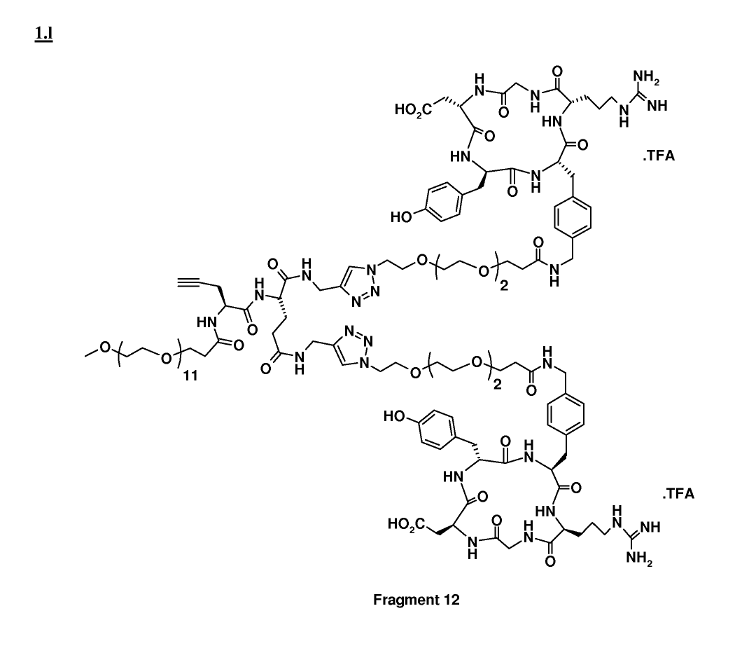

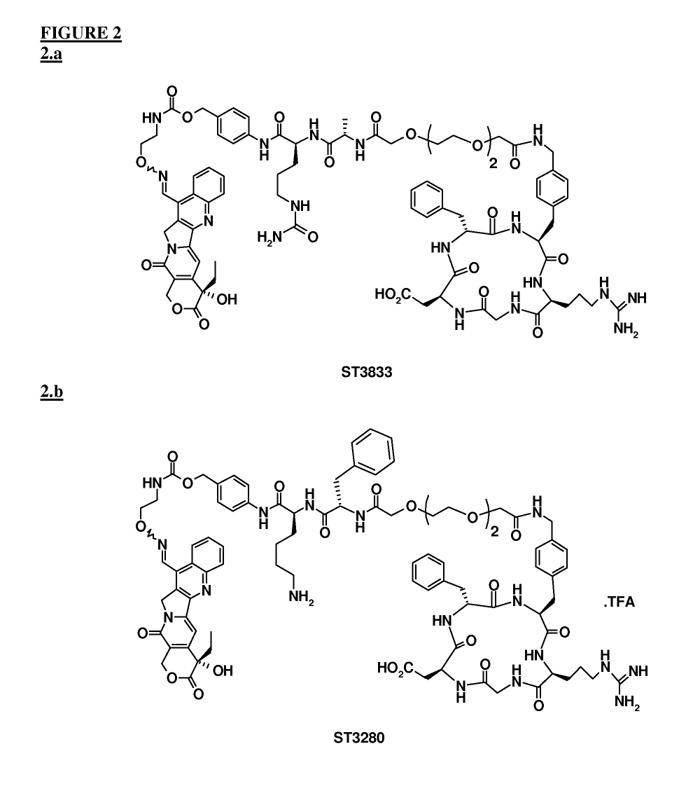

The following illustrated Examples are by no means an exhaustive list of what the present invention intends to protect.

### EXAMPLES

### Abbreviations

Aad: aminoadipic acid
Alloc: allyloxycarbonyl
Amp: p-aminomethyl phenylalanine
Boc: t-butoxycarbonyl
Cit: citrulline
CPT: camptothecin
DCM: dichloromethane
DIPEA: diisopropylethylamine
DMF: dimethylformamide
equiv.: equivalent
Et₂O: diethyl ether

### Fmoc: 9H-fluorenylmethoxycarbonyl

HCTU: (2-(6-chloro-1H-benzotriazole-1-yl)-1,1,3,3-tetramethylaminium hexafluorophosphate)
HOAt: 1-hydroxy-7-azabenzotriazole
HOBt: 1-hydroxybenzotriazole
MALDI: matrix assisted laser desorption ionization
MeOH: methanol

### NMP: N-methylpyrrolidone

PABA: 4-aminobenzylalcohol
PABC: para-aminobenzyloxycarbonyl
Pmc: 2,2,5,7,8-pentamethyl-chromane-6-sulfonyl
RP-HPLC: reversed phase-high-performance liquid chromatography
RT: room temperature
rt: retention time
SPPS: solid-phase peptide synthesis
TBTU: O-(benzotriazol-1-yl)-N,N,N′N′-tetramethyluronium tetrafluoroborate
TEA: triethylamine
TFA: trifluoroacetic acid
Tof: time of flight

General Remarks: ¹H spectra were recorded in DMSO-D₆, CDCl₃, or D₂O solution as indicated, at 300 MHz with a Bruker instrument. The chemical shift values are given in ppm and the coupling constants in Hz. Flash column chromatography was carried out using silica gel (Merck 230-400 mesh).

### Example 1

### Synthesis of ST3833

Fragment 2 (1 equiv) dissolved in 2 ml of DMF was added to a DMF (7 ml) solution containing Fragment 1, (prepared in situ, 0.32 mmol) and DIPEA (1 equiv). pH was adjusted to about 7.5 with DIPEA, and the reaction mixture was stirred at RT in darkness. After 2 h, a further equivalent of Fragment 1 was added, again adjusting the pH and the reaction mixture left under stirring overnight.

After purification by preparative HPLC (column, Discovery Bio Wide pore C18, Supelco, 250×21.2 mm, 10 μm; mobile phase: 29% CH₃CN in H₂O+0.1% TFA, λ=220 nM) and freeze drying, 365 mg of ST3833 were obtained with 97.6% purity.

Yield 60%.

Analytical HPLC (Gemini, Phenomenex, C18, 250×4.6 mm, 5 μm; mobile phase: 34% CH₃CN in H₂O+0.1% TFA, λ=220 nm). The conjugate shows two peaks at rt 7.96 and 10.43 min, due to the mixture of the E/Z isomers of the original cytotoxic molecule.

Maldi-Tof mass: 1650.71 [M+H]⁺.

¹H-NMR (DMSO-D₆), main shifts, δ: 9.28, 8.57, 8.28, 8.22, 8.14, 8.07, 7.93, 7.88, 7.75, 7.65, 7.55, 7.45, 7.36, 7.24, 7.15, 7.11, 7.03, 7.02, 6.42, 5.95, 5.42, 4.94, 4.60, 4.41, 4.28, 4.09, 3.95, 3.89, 3.57, 3.48, 3.18, 3.00-2.31, 1.91, 1.75, 1.60-1.30, 1.25, 0.90.

### Example 2 (for Comparison)

### Synthesis of ST3280

Coupling between Fragment 1 and Fragment 3 was performed following the procedure described in example 1 prior to removal of the alloc protecting group. To a solution of [Alloc-ST3280], (0.078 mmol) in 3 ml of DMF, were added Bu₃SnH (0.172 mmol), AcOH (0.375 mmol) and Pd(PPh₃)₄ (0.003 mmol). The reaction mixture was stirred for 1 h at RT under Ar. After evaporation of the solvent under reduced pressure, the residue was purified by preparative HPLC (column, Alltima, Alltech, RP18, 10 μm, 250×22 mm; mobile phase: 34% CH₃CN in H₂O+0.1% TFA). After freeze drying, the conjugate was obtained in 99.9% purity.

Yield=55%.

Analytical HPLC (Gemini, Phenomenex, C18, 250×4.6 mm, 5 μm; mobile phase: 35% CH₃CN in H₂O+0.1% TFA; λ=360 nm). rt of the E/Z isomers: 7.24 and 9.61 min.

ESI mass: 1696 [M+H]⁺.

¹H-NMR (DMSO-D₆), main shifts, δ: 8.57, 8.28, 8.22, 8.14, 8.07-7.50, 7.36, 7.24, 7.20-6.90, 6.42, 5.42, 4.94, 4.60, 4.41, 4.28, 4.18-4.00, 3.95, 3.90, 3.57, 3.48, 3.12-2.25, 1.91, 1.55, 1.38, 0.90.

### Example 3

### Synthesis of ST4167

To a solution of Fragment 4 (0.09 mmol) and Fragment 5 (88 mg, 0.09 mmol) in 2 ml of DMF, a solution of sodium ascorbate (0.089 mmol) and CuSO₄.5 H₂O (0.009 mmol) in 500 μl of H₂O was added. The pH was adjusted to pH 6 by addition of NaOH and the suspension was stirred at RT overnight. After evaporation of the solvent under reduced pressure, the residue was purified by preparative HPLC (column, Alltima C18, 10 μm, Alltech; mobile phase: 33% CH₃CN in H₂O+0.1% of TFA, λ=220 nm). After freeze drying, 72 mg of the desired adduct were obtained with 97% purity.

Yield=44%.

Analytical HPLC: (column, Gemini C18, 250×4.6 mm, 5 μm; mobile phase: 34% CH₃CN in H₂O+0.1% TFA, λ=220 nm). rt=7.7 and 9.9 min.

ESI mass: 1745.7 [M+H]⁺.

¹H-NMR (DMSO-D₆₊D₂O), main shifts, δ: 8.90, 8.44, 8.33, 8.18, 8.03-7.84, 7.8-7.69, 7.45, 7.39, 7.2-6.94, 5.48-5.30, 5.19, 4.89, 4.69, 4.6-4.24, 4.20, 4.13, 4.02, 3.89-3.52, 3.5-3.37, 3.24, 3.10-2.62, 2.40-2.30, 1.93-1.25, 0.85.

### Example 4

### Synthesis of ST4215

Coupling between Fragment 4 and Fragment 6 was performed following the procedure described in example 3.

The crude reaction product obtained from cycloaddition was purified by preparative HPLC (column, Alltima, C18, 10 μm, Alltech; mobile phase: 30% CH₃CN in H₂O+0.1% TFA). After freeze drying, 52 mg of the desired adduct were obtained with 98.6% purity.

Yield=41%.

Analytical HPLC (Gemini, Phenomenex, C18, 250×4.6 mm, 5 μm; mobile phase: 30% CH₃CN in H₂O+0.1% TFA, λ=220 nm). rt=11.23 and 15.43 min.

ESI mass: 2106 [M+H]⁺.

¹H-NMR (DMSO-D₆), main shifts, δ: 9.79, 9.13, 8.42, 8.15, 7.95, 7.86, 7.80-7.69, 7.45-7.39, 7.18-6.70, 5.47-5.24, 4.85, 4.60-4.30, 4.28-3.65, 3.64-3.31, 3.30-2.61, 2.43-2.30, 1.91-1.38, 1.33, 0.84.

### Example 5

### Synthesis of ST5548TF1

The cycloaddition between Fragment 4 and Fragment 7 was performed following the procedure described in Example 3. After preparative HPLC, the desired adduct was obtained with 100% purity.

Yield=45%.

Analytical HPLC (Gemini, Phenomenex, C18, 250×4.6 mm, 5 μm; mobile phase: 29% CH₃CN in H₂O+0.1% TFA, λ=220 nm). rt=10.84 and 15.22 min.

Maldi mass: 2120.89 [M+H]⁺.

¹H-NMR (DMSO-D₆) main shifts, δ: 9.94, 9.28, 9.04, 8.58, 8.52, 8.27-8.17, 8.03, 7.93-7.73, 7.55, 7.37, 7.25, 7.11-7.07, 6.82, 6.56, 6.41, 5.90, 5.42-5.29, 4.95, 4.60-4.53, 4.46, 4.37, 4.25, 4.16, 4.01-3.96, 3.84, 3.65-3.37, 3.17, 3.10, 3.01-2.88, 2.42-2.36, 1.90-1.86, 1.75-1.71, 1.61-1.58, 1.50-1.30, 0.89.

### Example 6

### Synthesis of ST5546TF1

Coupling between Fragment 4 and Fragment 8 was performed following the procedure described in Example 3. The crude reaction product obtained from cycloaddition was purified by preparative HPLC (Alltima, Alltech, RP18, 250×22 mm, 10 μm; mobile phase: 28% CH₃CN in H₂O+0.1% TFA, λ=220 nm). After freeze drying, ST5546TF1 was obtained with 100% purity.

Yield=38%.

Analytical HPLC (Gemini, Phenomenex, C18, 250×4.6 mm, 5 μm; mobile phase: 28% CH₃CN in H₂O+0.1% TFA, λ=220 nm). rt=11.38 and 16.16 min.

Maldi mass: 2480 [M+H]⁺.

¹H-NMR (D₂O) main shifts, δ: 8.73, 8.52, 7.83-7.74, 7.62, 7.39, 7.19, 7.05, 6.93, 6.87, 6.63, 5.58-5.49, 4.91, 4.68-4.26, 4.04, 3.85-3.42, 3.24-3.12, 2.93-2.87, 2.77, 2.65-2.60, 2.11, 1.93, 1.82, 1.72, 1.63, 1.58-1.49, 1.12.

### Example 7

### Synthesis of ST5744TF1

A 14 μl aqueous solution of sodium ascorbate (0.014 mmol) and of CuSO₄.5H₂O (0.0014 mmol) were added to a 2 ml solution (DMF/H₂O: 1/1) containing Fragment 9 (15 mg, 0.014 mmol) and Fragment 10 (34 mg, 0.016 mmol). The resulting reaction mixture was stirred at RT for 1.5 h. Solvent was then removed under reduced pressure. After purification through HPLC (column, Alltima, Alltech, C18, 10 μm, 250×22 mm; mobile phase: 30% CH₃CN in H₂O+0.1% TFA), the desired adduct was obtained.

Yield=37%.

Analytical HPLC (column Gemini, mobile phase 29% CH₃CN in H₂O+0.1% TFA). rt=9.2 and 12.6 min.

Maldi-TOF [M+H]⁺2988.78.

¹H-NMR (DMSO-D₆+D₂O) main shifts, δ: 9.30, 8.56, 8.40, 8.22, 8.19, 8.01, 7.92-7.85, 7.83, 7.78-7.69, 7.53, 7.37, 7.23, 7.08, 6.68, 5.42-5.3, 5.21, 5.10, 4.93, 4.74, 4.37-4.34, 4.23, 4.20-4.03, 3.89, 3.85, 3.61, 3.56-3.36, 3.29-3.16, 3.07, 3.00-2.73, 2.38, 2.10, 1.85, 1.72, 1.55, 1.40-1.30, 1.23, 0.87.

### Example 8

### Synthesis of ST5745TF1

A 16 μl aqueous solution of sodium ascorbate (0.016 mmol) and of CuSO₄.5H₂O (0.0016 mmol) were added to a solution (DMF/H2O: 4/3, 3.5 ml) containing Fragment 11 (33.2 mg, 0.032 mmol) and Fragment 12 (84 mg, 0.031 mmol). The resulting solution was submitted to microwaves irradiation (90 W) for 2 min. The maximum temperature observed reached 120° C. After purification through HPLC (column, Alltima, Alltech, C18, 10 μm, 250×22 mm; mobile phase: 32% CH₃CN in H₂O+0.1% TFA), the desired adduct was obtained with 97% purity.

Yield=42%.

Analytical HPLC (column Gemini, mobile phase 29% CH₃CN in H₂O+0.1% TFA). rt=10.2 and 12.5 min.

Maldi mass: [M+H]⁺ 3723.

¹H-NMR (DMSO-D₆+D₂O) main shifts, δ: δ: 9.05, 8.34-8.09, 7.82-7.71, 7.42-7.24, 7.06-6.99, 6.66, 5.49, 5.55-5.11, 4.79, 4.57, 4.37-3.97, 3.70-3.38, 3.16, 3.01-2.87, 2.34-2.32, 2.00-1.55, 1.42-1.28, 1.19, 0.84.

### Example 9

### Synthesis of Fragment 1

### c{Arg-Gly-Asp-D-Phe-Amp [CO—CH₂—(O—CH₂—CH₂)₂—O—CH₂—CO—N₃]}

### Microwave Assisted Solid Phase Synthesis of Cyclopeptide Acylhydrazide

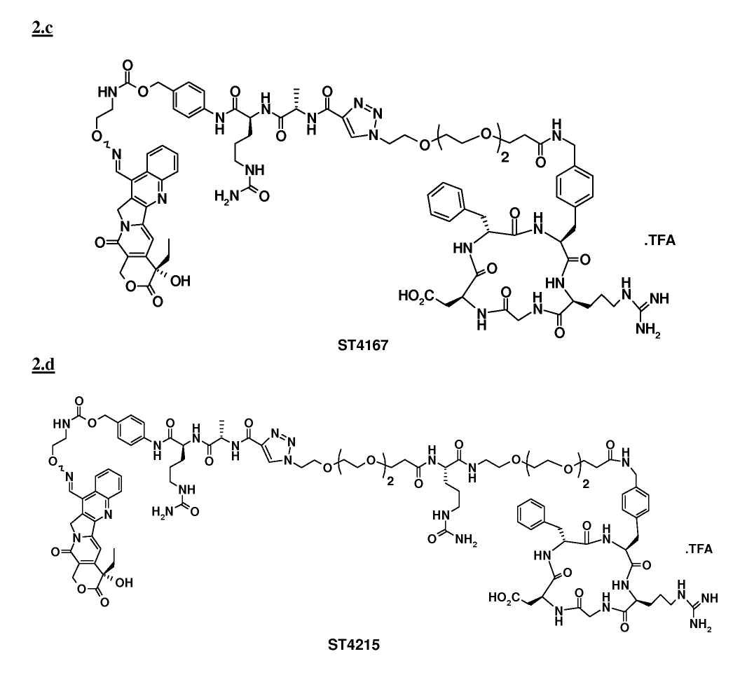

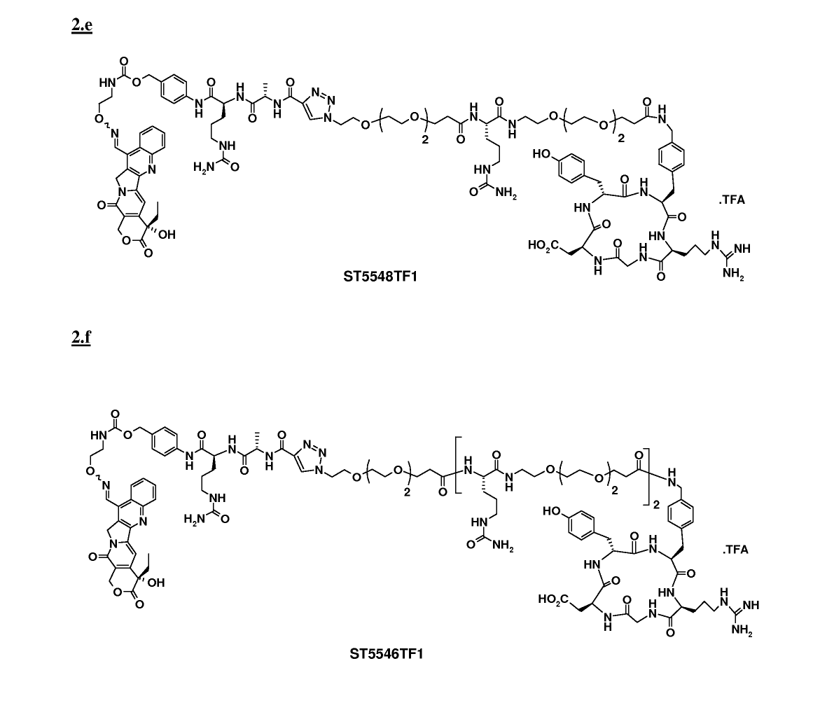

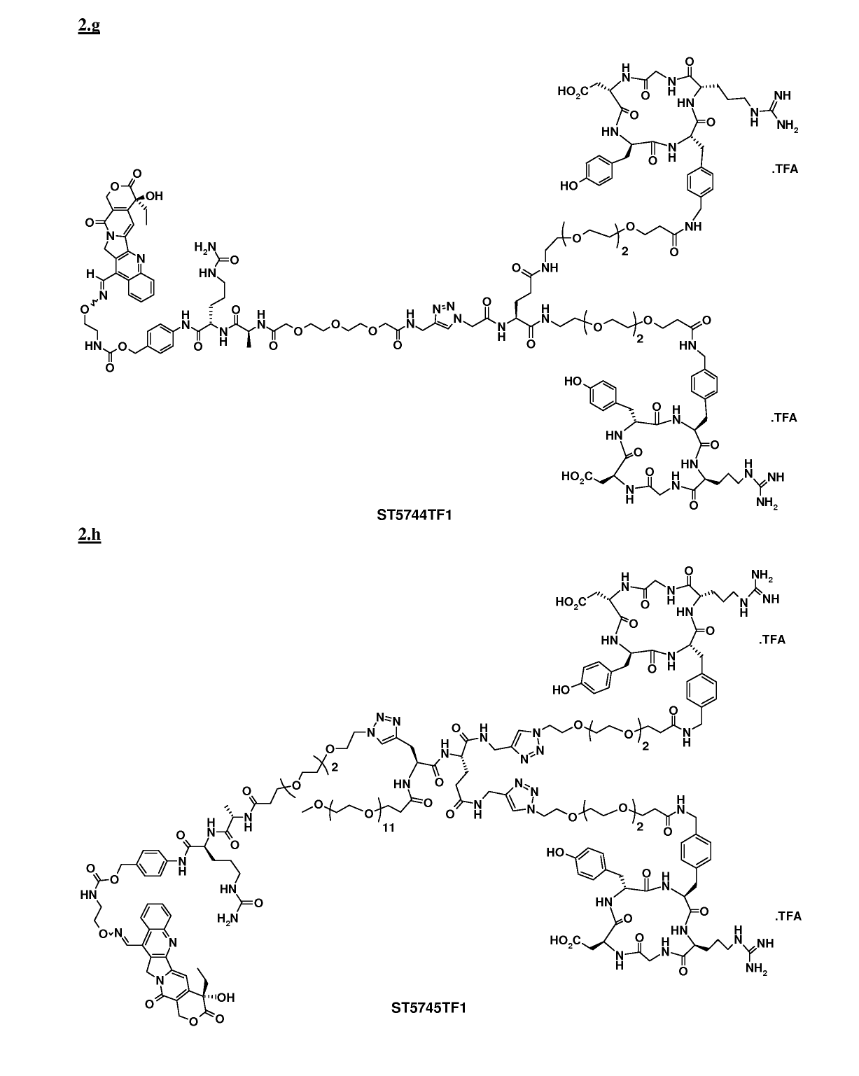

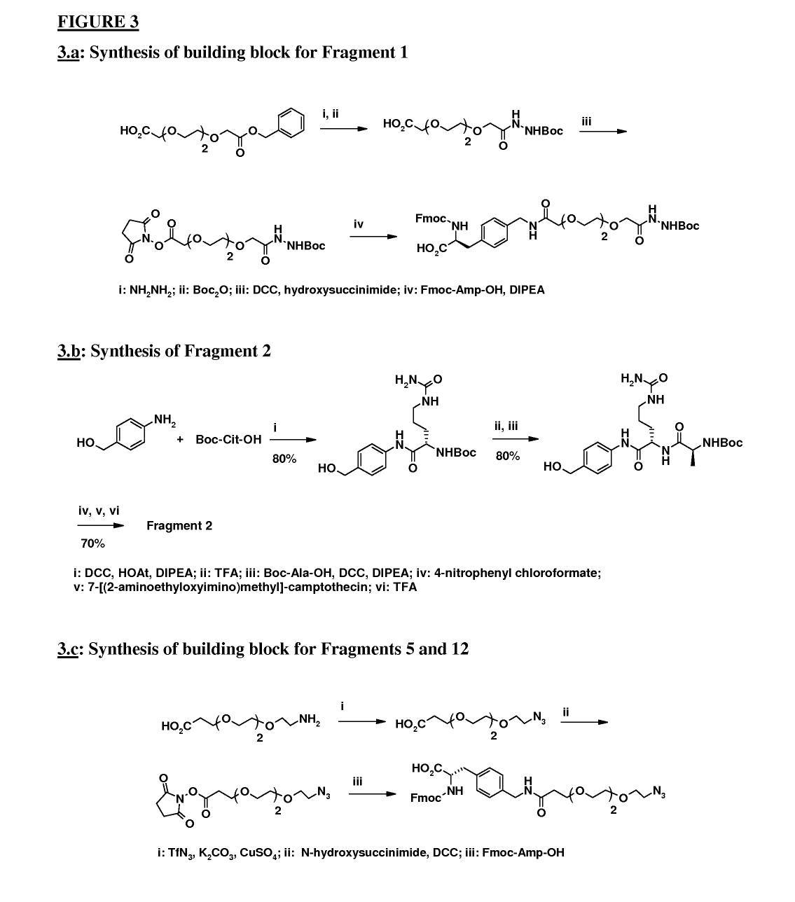

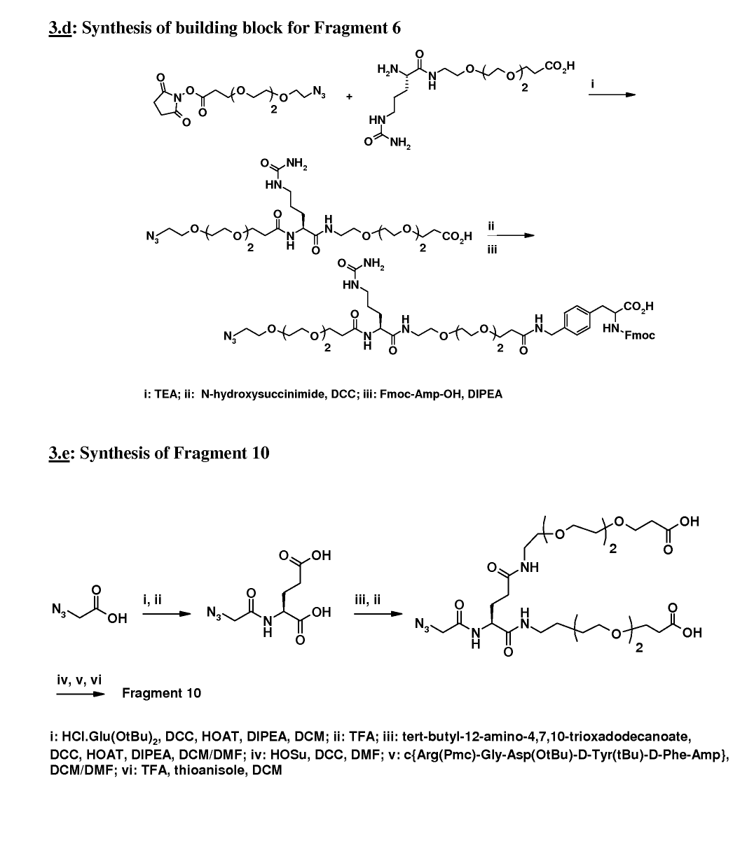

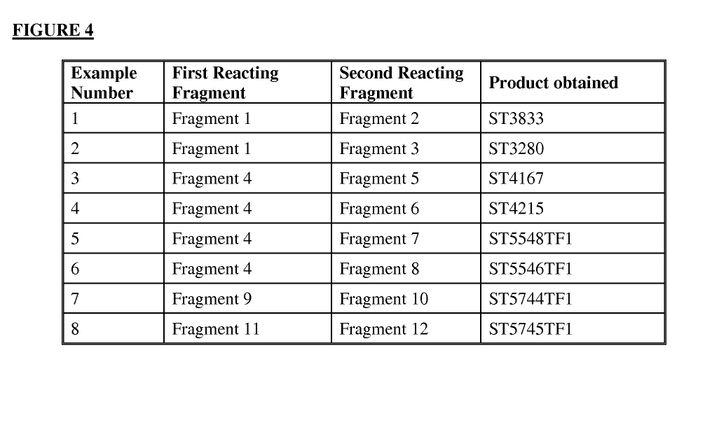

Fmoc-Gly-SASRIN® (2.53 g, 2 mmol) was suspended in 40 ml of DMF containing 20% piperidine and submitted to 25 W for 3 min. After filtration and washing of the resin, a solution containing 2 equiv. of the next amino acid was added followed by addition of a solution containing 2 equiv. of HOBT and TBTU in 36 ml of DMF. Finally, 4 equiv. of DIPEA dissolved in 5 ml of NMP were added and the suspension was irradiated at 30 W for 5 min. After filtration and Fmoc deprotection, the next couplings were carried out in the same way until the peptide was completed. The order of addition of the amino acids was Fmoc-Arg(Pmc)-OH, Fmoc-Amp building block (see FIG. 3a for the synthesis), Fmoc-D-Phe-OH and Fmoc-Asp(OtBu)-OH.

After the last Fmoc deprotection and washing, cleavage from the resin was performed by treatment with a 1% solution of TFA in DCM (60 ml) for 15 min. After filtration, the same operation was repeated for 5 times. The combined filtrates were neutralized by addition of pyridine and taken to dryness. To the residue dissolved in 1500 ml of CH₃CN, HOBT and TBTU (3 equiv) plus 1% DIPEA were added and the reaction mixture was stirred for 1 h at RT. The solvent was then evaporated under reduced pressure. After purification by flash chromatography (DCM/MeOH: 94/6→92/8) the desired protected cyclopeptide was obtained in 50% yield.

The latter was dissolved in TFA/H₂O: 95/5 and stirred at RT for 1 h. The solvent was then evaporated under reduced pressure and the cyclopeptide was obtained in 98% yield after purification by precipitation from TFA/Et₂O. Analytical HPLC (column, Purosphere STAR® Merck, RP18, 250×4 mm, 5 μm; mobile phase: 20% CH₃CN in H₂O+0.1% TFA; λ=220 nm). rt=9.14 min.

Maldi-Tof mass: 870.13 [M+H]⁺.

The deprotected acylhydrazide (0.32 mmol) and HOAT (1.91 mmol) were dissolved in 7 ml of DMF and t-butyl nitrite (0.38 mmol) was added. The reaction mixture was stirred for 30 min. The acyl azide was not isolated and was used without any purification in the next step.

### Example 10

### Synthesis of Fragment 2

### HCl.Ala-Cit-PABC-CPT

### Step 1:

A solution of Boc-Cit-OH (1 g, 3.63 mmol), (PABA, 1.3 g, 10.9 mmol), HOAT (0.74 g, 5.45 mmol), DIPEA (0.93 ml, 5.45 mmol) and DCC (1.12 g, 5.45 mmol) in DMF (65 ml) was stirred at RT overnight. After evaporation of the solvent at reduced pressure, the residue was purified by flash chromatography (DCM/MeOH: 90/10→85/15). Boc deprotection was performed by reacting the former intermediate with TFA/DCM: 1/1; affording after removal of the solvent under reduced pressure, 520 mg of TFA.Cit-PABA.

Yield=73%.

### Step 2:

To a solution of Alloc-Ala-OH (472 mg, 2.68 mmol), DCC (272 mg, 1.34 mmol) and DIPEA (460 μl, 2.68 mmol) in a mixture of DCM/DMF (v/v=1/1, 20 ml) of at 0° C. was added TFA.Cit-PABA and the solution left under stirring for 6 h. The solvent was removed under reduced pressure and the residue was dissolved in water at pH 2. The resulting solution was extracted twice with EtOAc. The aqueous phase was neutralized by addition of NaHCO₃ and water was removed under reduced pressure. Purification by flash chromatography (EtOAc/MeOH=85/15), gave 398 mg of Alloc-Ala-Cit-PABA.

Yield=69%.

### Step 3:

To a solution of the latter (392 mg, 0.9 mmol) in 5 ml of dry DMF, 4-nitro-phenyl chloroformate (363 mg, 1.8 mmol) dissolved in 20 ml of DCM and 150 μl of pyridine were added and the reaction mixture was stirred for 1 h. The solvent was removed under reduced pressure and the residue was triturated several times with cold Et₂O.

### Step 4:

To a solution of the previous adduct in 25 ml of DMF, were added 7-(2-aminoethoxyimine)-methyl-camptothecin.HCl (423.5 mg, 0.90 mmol) and TEA (150 μl, 1.1 mmol) and the reaction mixture was stirred for 5 h. The solvent was removed under reduced pressure and the residue was triturated several times with water to remove excess of TEA. After purification by flash chromatography (DCM/MeOH: 90/10), 320 mg (0.36 mmol) of the protected Fragment 2 were obtained.

Yield=40% (2 steps).

### Step 5:

To a solution of the above obtained protected Fragment 2 in DMF (3.8 ml) were added a solution of Bu₃SnH (220 μl, 0.8 mmol) in DCM (3.8 ml), 40 μl of water and finally Pd[(PPh)₃]₄ (17 mg, 0.014 mmol) and the resulting reaction mixture was stirred for 15 min. The solvent was removed under reduced pressure to yield a solid that was taken up in water (65 ml) at pH 3. The aqueous layer was extracted with Et₂O (25 ml×3) before being concentrated to give the pure Fragment 2 as its hydrochloride salt.

Yield=93%.

HPLC (Gemini, Phenomenex, C18, 250×4.6 mm, 5 μm; mobile phase: 28% CH₃CN in H₂O+0.1% TFA, λ=220 nm). rt=8.9 and 12.3 min. Maldi mass=834 [M+Na]⁺.

### Example 11

### Synthesis of Fragment 3

### TFA.Phe-Lys(Alloc)-PABC-CPT

The title compound was obtained following the procedure described in example 10 starting from Boc-Lys(Alloc)-OH instead of Boc-Cit-OH and using Boc-Phe-OH in the second step instead of Alloc-Ala-OH.

Analytical HPLC (Purosphere STAR, Merck, 5 μm; mobile phase: 35% CH₃CN in H₂O+0.1% TFA, λ=220 nm). rt=18.00 and 25.29 min.

Maldi mass: 965 [M+Na]⁺.

### Example 12

### Synthesis of the Fragment 4 (HC≡C—CO-Ala-Cit-PABC-CPT)

To a solution of Fragment 2 (0.12 mmol) in 3 ml of DMF, DIPEA (0.31 mmol), propiolic acid (0.18 mmol) and HOAT (0.18 mmol) were added and the solution was cooled at 0° C. before adding DCC (0.21 mmol). The reaction mixture was stirred at RT for 1.5 h. After removal of the solvent under reduced pressure, the residue was purified by flash chromatography (DCM/MeOH:9/1→8/2).

Yield=72%.

Analytical HPLC (Gemini, Phenomenex, C18, 250×4.6 mm, 5 μm; mobile phase: 31% CH₃CN in H₂O+0.1% TFA, λ=220 nm). rt=11.46 and 16.14 min.

Maldi mass: 863.8 [M+H]⁺ and 885.8 [M+Na]⁺.

### Example 13

### Synthesis of Fragment 5 c{Arg-Gly-Asp-D-Phe-Amp-[CO—(CH₂)₂—(O—CH₂—CH₂)₂—O—(CH₂)₂—N₃]}

The title cyclopeptide was synthesized following the procedure described in Example 9, incorporating the building block Fmoc-Amp[CO—(CH₂)₂—(O—CH₂—CH₂)₂—O—(CH₂)₂—N_{3]} at the second step of SPPS.

Analytical HPLC (Gemini, Phenomenex, C18, 250×4.6 mm, 5 μm; mobile phase: 30% CH₃CN in H₂O+0.1% TFA, λ=220 nm). rt=8.3 min.

Maldi mass: 881 [M+H]⁺.

### Example 14

### Synthesis of Fragment 6 c{Arg-Gly-Asp-D-Phe-Amp-[CO—(CH₂)₂—(O—CH₂—CH₂)₂—O—(CH₂)₂—H-Cit-CO—(CH₂)₂—(O—CH₂—CH₂)₂—O—(CH₂)₂—N₃]}

This cyclopeptide was synthesized following the procedure described in Example 9 incorporating the building block Fmoc-Amp-[CO—(CH₂)₂—(O—CH₂—CH₂)₂—O—(CH₂)₂—NH-Cit-CO—(CH₂)₂—(O—CH₂—CH₂)₂—O—(CH₂)₂—N₃]} instead of Fmoc-Amp [CO—CH₂—(O—CH₂—CH₂)₂—O—CH₂—CO—N₃] at the second step of SPPS. Analytical HPLC (Gemini, Phenomenex, C18, 250×4.6 mm, 5 μm; mobile phase: 25% CH₃CN in H₂O+0.1% TFA). rt=10.79 min.

Maldi-Tof mass: 1241 [M+H]⁺.

### Example 15

### Synthesis of Fragment 7

### c{Arg-Gly-Asp-D-Tyr-Amp[CO—(CH₂)₂—(O—CH₂—CH₂)₂—O—(CH₂)₂—NH-Cit-CO—(CH₂)₂—(O—CH₂—CH₂)₂—O—(CH₂)₂—N₃]}

The title cyclopeptide was synthesized following the procedure described in Example 14, incorporating Fmoc-D-Tyr-(t-Bu)-OH at the third step of SPPS. Analytical HPLC (Gemini, Phenomenex, C18, 250×4.6 mm, 5 μm; mobile phase: 22% CH₃CN in H₂O+0.1% TFA, λ=220 nm). rt=8.87 min.

Maldi mass: 1256.96 [M+H]⁺.

¹H-NMR (D₂O) main shifts, δ: 7.43, 7.29, 7.19, 6.94, 4.93, 4.59, 4.53-4.37, 4.01-3.65, 3.57, 3.35, 3.27, 3.14-3.07, 2.95-2.87, 2.79-2.72, 2.03-1.60.

### Example 16

### Synthesis of Fragment 8

### c{Arg-Gly-Asp-D-Tyr-Amp[CO—(CH₂)₂—(O—CH₂—CH₂)₂—O—(CH₂)₂NH-Cit]₂CO—(CH₂)₂—(O—CH₂—CH₂)₂—O—(CH₂)₂N₃}

The title cyclopeptide was synthesized following the procedure described in Example 15, incorporating Fmoc-Amp-[CO—(CH₂)₂—(O—CH₂—CH₂)₂—O—(CH₂)₂NH-Cit]₂-CO—(CH₂)₂—(O—CH₂—CH₂)₂—O—(CH₂)₂N₃ at the second of the SPPS.

Analytical HPLC (Gemini, Phenomenex, C18, 250×4.6 mm, 5 μm; mobile phase: 21% CH₃CN, λ=220 nm). rt=11.62 min.

Maldi mass: 1617.31 [M+H]⁺.

¹H-NMR (D₂O), main shifts, δ: 7.23, 7.10, 7.05, 6.73, 4.58, 4.40, 4.33-4.17, 3.82-3.47, 3.40, 3.38, 3.16-3.05, 2.96-2.82, 2.75, 2.69, 2.58, 1.84-1.40.

### Example 17

### Synthesis of Fragment 9

### Step 1:

To a suspension of anhydrous 3,6,9-trioxaundecanedioic acid (2.1 g, 9.43 mmol) in 63 ml DCM at 0° C., DCC (97.2 mg, 0.47 mmol), p-nitrophenol (437 mg, 0.31 mmol), TEA (1.31 ml, 9.43 mmol) and DMAP (7.7 mg, 0.06 mmol) were added. After 30 min the reaction mixture was washed with H₂O, 0.1 N HCl, H₂ and, after drying over sodium sulfate, concentrated to small volume and kept in freezer for 1 h. before being filtered. Propargylamine hydrochloride (144 mg, 1.57 mmol) and TEA (262 μl, 1.88 mmol) were added to the filtrate and after a few minutes the solvent was removed under reduced pressure. The resulting residue was dissolved in 20 ml of H₂O and filtered through Dowex 50 W X8. The mother liquors were extracted twice with DCM to remove the remaining nitrophenol and concentrated to afford the desired alkyne-PEG-CO₂H as a white solid.

Yield=72%.

### Step 2:

DCC (25 mg, 0.12 mmol) was added to a cold (0° C.) solution of Ala-Cit-PABC-CPT (Fragment 2, 56 mg, 0.06 mmol), alkyne-Peg-CO₂H (22 mg, 0.085 mmol), HOAT (16 mg, 0.12 mmol) and DIPEA (41 μl, 0.24 mmol) in 1.5 ml DMF. The reaction mixture was then stirred at RT overnight. After filtration, the filtrate was concentrated to dryness and the resulting residue was purified by flash chromatography (DCM/MeOH:85/15) to finally obtain 40 mg of the desired adduct as a yellow solid.

Yield=63.5%.

Analytical HPLC (column Gemini Phenomenex C18; 250×4.6 mm, 5 μm; 32% CH₃CN in H₂O+0.1% TFA). rt=11.6 and 16.3 min.

ESI mass [M+H]⁺ 1053.42

### Example 18

### Synthesis of Fragment 10 (See FIG. 3e)

### Step 1:

DCC (84 mg, 0.41 mmol) was added to a cold (0° C.) solution of L-glutamic acid di-tert-butyl ester hydrochloride (100 mg, 0.34 mmol), azidoacetic acid (41 mg, 0.41 mmol), HOAT (0.41 mmol) and DIPEA (127 ml, 0.74 mmol) in 4.6 ml DCM. The reaction mixture was stirred at RT for 2.5 h. After filtration, the organic solution was diluted with DCM up to 30 ml and washed with H₂O, 1 N HCl, 5% NaHCO₃ and H₂O. The solvent was removed under reduced pressure and the resulting residue was dissolved in 3 ml TFA and stirred for 1 h. TFA was removed in its turn under reduced pressure to afford 2-(2-azido-acetylamino)-pentanedioic acid.

### Step 2:

2-(2-Azido-acetylamino)-pentanedioic acid was dissolved in a 45 ml mixture of DCM/DMF (8/1). Standard coupling with tert-butyl-12-amino-4,7,10-trioxadodecanoate (281 mg, 1.01 mmol), HOAT (137 mg, 1.01 mmol), DIPEA (174 μl) and DCC (209 mg, 1.014 mmol) allowed the obtention of a crude product that was purified by flash chromatography (DCM/MeOH:95/5) to afford 175 mg of the desired bis-carboxylic ester intermediate as a solid product.

Yield=68.4%.

¹H-NMR (CDCl₃), δ: 7.54, 7.23, 6.74, 4.42, 4.01, 3.70, 3.61, 3.41, 2.50, 2.35, 2.08, 1.44.

### Step 3:

The above obtained compound was deprotected using standard conditions by means of TFA. Once all the starting material disappeared, TFA was removed under reduced pressure to lead to the bis-carboxylic intermediate that was used in the next step without any further purification.

### Step 4:

A solution of N-hydroxysuccinimide (63 mg, 0.55 mmol) in DMF was added at 0° C. to a solution of the above obtained intermediate, followed by addition of DCC (115 mg, 0.55 mmol). The reaction mixture was stirred overnight at RT. The crude desired product was obtained after a standard work-up, and used in the next step without any further purification.

### Step 5:

The above obtained intermediate was dissolved in 2 ml DCM and reacted at RT for 1.5 h with cyclopeptide c{Arg(Pmc)-Gly-Asp(OtBu)-D-Tyr(tBu)-Amp} (725 mg, 0.69 mmol) dissolved with 3.5 ml DMF in the presence of DIPEA (153 μl, 0.93 mmol). The cyclopeptide c{Arg(Pmc)-Gly-Asp(OtBu)-D-Tyr(tBu)-Amp} was prepared by SPPS according to the procedure described in example 15 using Fmoc-Amp(Cbz)-OH instead of Fmoc-Amp-[CO—(CH₂)₂—(O—CH₂—CH₂)₂—O—(CH₂)₂—NH-Cit-CO—(CH₂)₂—(O—CH₂—CH₂)₂—O—(CH₂)₂—N₃]}). The crude residue was purified by preparative HPLC (column Alltima, C18 Alltech; 10 μm, 250×22 mm; 69% CH₃CN in H₂O+0.1% TFA).

Yield=48%.

### Step 6:

Final deprotection was performed in 1 ml DCM with TFA (540 equiv) and thioanisole (110 equiv) to afford the crude product that was purified via several successive precipitations from cold Et₂O. The desired fragment 10 was obtained as a white solid.

Yield=69%.

Analytical HPLC (column Gemini Phenomenex C18; 250×4.6 mm, 5 μm; 22% CH₃CN in H₂O+0.1% TFA). rt=10.9

MALDI mass [M+H]⁺1935.22.

### Example 19

### Synthesis of Fragment 11

To a solution of Fragment 2 (80 mg, 0.094 mmol) and DIPEA (19 μl, 0.11 mmol) in DMF (1 ml), was added the succinimide derivative obtained in step ii during the synthesis of the building block for Fragment 5 (FIG. 3c, 37 mg, 0.11 mmol) dissolved in 0.5 ml DCM. The reaction mixture was stirred at RT for 5 h. After evaporation of the solvent under reduced pressure, the residue was purified by preparative HPLC (column Alltima, 10 μm, 250×22 mm; mobile phase 37% CH₃CN in H₂O+0.1% TFA). rt=9.7 and 12.4 min.

Yield=76.3%.

ESI mass [M+H]⁺ 1041.42.

### Example 20

### Synthesis of Fragment 12

### Step 1:

An aqueous 2.5 M solution of sodium ascorbate (90 μl) and of 0.5 M CuSO₄.5H₂O (45 μl) was added to a 12 ml solution of DMF/H₂O (7/5) of (1,3-bis-prop-2-ynylcarbamoyl-propyl)-carbamic acid benzyl ester (72.5 mg, 0.20 mmol) and c {Arg(Pmc)-Gly-Asp(OtBu)-D-Tyr(tBu)-Amp-[CO—(CH₂)₂—(O—CH₂—CH₂)₂—O—(CH₂)₂—N₃]} (572 mg, 0.45 mmol). The latter was synthesized according to the procedure described at example 13 using Fmoc-D-Tyr-(t-Bu)—OH instead of Fmoc-D-Phe-OH. The resulting reaction mixture was submitted to microwaves irradiation (90 W) for 2 min. The maximum temperature observed was 121° C. The irradiation was repeated three times until complete disappearance of the starting material, which was monitored by HPLC (column Gemini, 250×4.6 mm, 5 μm; mobile phase 35% CH₃CN in H₂O+0.1% TFA). Solvent was then removed under reduced pressure and the crude reaction mixture was purified through flash chromatography (DCM/MeOH gradient: 93/7→90/10→80/20) to obtain 417 mg of the desired product.

Yield=71%.

ESI mass: 1453.6 (m/z 2+), 969.4 (m/z 3+).

### Step 2:

406 mg of the above obtained product dissolved in a mixture of DMF (3 ml) and MeOH (5 ml) was deprotected, in order to remove the benzyloxycarbonyl protecting group, by means of ammonium formate (44 mg, 0.70 mmol) and Pd/C (200 mg). The suspension was stirred for 3 h and then filtered. The solvent was removed under reduced pressure and the resulting product was used without any further purification in the next step.

### Step 3:

A solution of the above obtained product in DMF (3 ml) was added into a solution of the intermediate obtained from standard coupling between propargyl glycine and methyl-(PEG)12-NHS (102 mg, 0.15 mmol) in DCM (4.5 ml), followed by addition of HCTU (62 mg, 0.15 mmol) and DIPEA (51 μl, 0.30 mmol). The resulting solution was stirred at RT for 2 h. After removal of the solvent under reduced pressure, the residue was dissolved in DCM (300 ml) and washed with H₂O. The organic phase was then evaporated to afford 312 mg of the desired adduct.

Yield=67%.

ESI mass: 1741 (m/z 2+), 1168 (m/z 3+).

### Step 4:

The above obtained intermediate was fully deprotected by means of a mixture of TFA/DCM/thioanisole (1/1/0.3). The compound was purified by precipitation from cold Et₂O, to afford 245 mg of the desired Fragment 12.

Yield=92%.

MALDI mass: 2679.79 found.

### Biological Results

### Solid-Phase Binding Assay of the Conjugates to Integrin Receptors αᵥ β₃ and αᵥ β₅

The receptor binding assays were performed as described (Orlando R. A., et al., J. Biol. Chem. 1991, 266, 19543). αᵥβ₃ and αᵥβ₅ were diluted to 500 ng/ml and 1 μg/ml, respectively, in coating buffer (20 mM Tris, pH 7.4, 150 mM NaCl, 2 mM CaCl₂, 1 mM MgCl₂, 1 mM MnCl₂) and an aliquot of 100 μL was added to a 96-well microtiter plate and incubated overnight at 4° C. The plate was washed once with blocking/binding buffer (50 mM Tris, pH 7.4, 100 mM NaCl, 2 mM CaCl₂, 1 mM MgCl₂, 1 mM MnCl₂, 1% bovine serum albumin), and then was incubated for additional 2 h at RT The plate was rinsed twice with the same buffer and incubated for 3 h at RT with radiolabeled ligand [¹²⁵I]Echistatin (Amersham Pharmacia Biotech) 0.05 nM (0.1 nM for αᵥ β₅) in the presence of competing inhibitors. After the incubation, the wells were washed and radioactivity was determined with a gamma-counter (Packard). Non-specific binding of ligand was determined with molar excess (200 nM) of cold echistatin.

The IC₅₀ values reported in Tables 1 and 2 were calculated as the concentrations of compounds required for 50% inhibition of echistatin binding and were estimated by the Prism GraphPad program. The Ki of the competing ligands were calculated according to the Cheng-Prusoff equation (Cheng Y. C., et al., Biochem. Pharmacol., 1973, 22, 3099). Values are the mean±log standard error of triplicate determinations from two independent experiments. Most of the conjugates showed a potent activity with inhibition in the low nanomolar range. It is noteworthy that the in vitro activity demonstrated by ST3280 was mainly due to the intrinsic activity of a decomposition product due to the instability of the compound itself.

TABLE 1
Inhibition of [¹²⁵I] Echistatin Binding to αᵥβ₃ receptor.

| Compound | IC₅₀ ± log SE (nM) | Ki (nM) |
|---|---|---|
| Echistatin | 0.28 ± 0.08 | 0.26 |
| ST3833 | 78.4 ± 1.5 | 61.0 |
| ST3280 | 9.7 ± 0.06 | 8.5 |
| ST4167 | 11.0 ± 0.8 | 8.7 |
| ST5744TF1 | 3.01 ± 0.11 | 2.4 |
| ST5745TF1 | 6.21 ± 0.09 | 4.92 |

TABLE 2
Inhibition of [¹²⁵I] Echistatin Binding to αᵥβ₅ receptors.

| Compound | IC₅₀ ± log SE (nM) | Ki (nM) |
|---|---|---|
| Echistatin | 0.29 ± 0.02 | 0.33 |
| ST3833 | 87.8 ± 1.21 | 68.2 |
| ST3280 | 34.4 ± 0.8 | 23.0 |
| ST4167 | 18.4 ± 0.89 | 13.8 |
| ST5744TF1 | 3.84 ± 0.12 | 2.95 |
| ST5745TF1 | 3.15 ± 0.11 | 2.41 |

### Adhesion Assay of Tumour Cells on Vitronectin

A2780 human ovarian carcinoma and PC3 prostate carcinoma cells were grown in medium culture RPMI 1640 containing 10% fetal bovine serum and 50 μg/ml gentamycin sulfate. Cells were maintained in a 37° C. incubator with saturated humidity and an atmosphere of 95% air and 5% CO₂. A2780 tumour cell line expresses high levels of αᵥβ₅ integrin, and PC3 low levels of both integrins.

In 96-well tissue culture plates, 50 μl/well of a solution of vitronectin (5 μg/ml) were added for 2 h at room temperature. The solutions were removed upsetting the plates. 50 μl/well of a solution 1% BSA were added for 1 h at RT. The plates were washed by addition of 100 μl/well of medium culture RPMI 1640 without fetal calf serum (FCS). The washing was repeated twice. The molecules were added at different concentrations in the range between 0.039 μM and 20 μM. The solutions were prepared by dilution 1:2 in medium culture without FCS. Tumour cells in the flasks were washed in saline solution before to be detached by scraper, by the addition of 5 ml of medium culture without FCS and 1% BSA. Tumour cells were counted after resuspension and added at an appropriate cellular density (40000-50000 cells/well). The plates were incubated for 1 h at 37° C. in humidified incubator with 5% CO₂. Then, the solutions were removed upsetting the plates and washed once with 200 μl/well of PBS with Ca²⁺ e Mg²⁺. Tumour cells were fixed with 100 μl of a solution 4% paraformaldehyde in 0.2 M Sorensen phosphate buffer pH 7.2-7.4 for 10 min at RT The plates were upset and 100 μl of 1% Toluidine Blu solution were added for 10 min at RT The plates were washed twice by immersion in bi-distilled water and then dried at 60° C. in thermostat incubator (Kottermann). 100 μl/well of 1% SDS were added. The plates were kept under stirring for 20 min at RT and were then evaluated by Victor 1420 multilabel counter (Wallac) at 600 nm.

The IC₅₀ value as parameter to measure the inhibiting effect of the molecules on tumour cell adhesion to vitronectin was evaluated using “ALLFIT” computer program.

The conjugates investigated were found to block tumour cells (PC3 and A2780) attachment to an extracellular matrix component such as vitronectin, the ligand of cell surface receptors integrin αᵥβ₃ and αᵥβ₅ with IC₅₀ values ranged from 0.39 to 4.6 μM (table 3) without showing an excessive selectivity on a tumour cell line. As mentioned for the binding affinity toward α_{V}β₃ receptors, ST3280 activity toward α_{V}β₅ receptors is the consequence of the cleavage of the compound and not of the compound itself.

TABLE 3
Antiadhesive effect of the conjugates on A2780 ovarian carcinoma cells and
PC3 prostate carcinoma cells to vitronectin (1 h of treatment)

| | PC3 | A2780 |
|---|---|---|
| Compounds | IC₅₀ ± SD (μM) | |
| ST3280 | 1.8 ± 0.4 | 2.7 ± 0.3 |
| ST3833 | 2.1 ± 0.2 | 0.9 ± 0.05 |
| ST4167 | 0.56 ± 0.07 | 0.67 ± 0.02 |
| ST4215 | 4.6 ± 0.8 | 1.7 ± 0.2 |
| ST5744TF1 | 0.39 ± 0.09 | 0.45 ± 0.02 |
| ST5745TF1 | 1.0 ± 0.05 | 1.0 ± 0.01 |

### Cytotoxicity of the Conjugates on Different Tumour Cell Lines

To evaluate the effect of the compound on survival cells, the sulphorodamine B test was used. To measure the effects of the compounds on cell growth, PC3 human prostate carcinoma and A2780 human ovarian carcinoma cells were used. A2780 and PC3 tumour cells were grown RPMI 1640 containing 10% fetal bovine serum (GIBCO).

Tumour cells were seeded in 96-well tissue culture plates at approximately 10% confluence and were allowed to attach and recover for at least 24 h. Varying concentrations of the drugs were then added to each well to calculate their IC₅₀ value (the concentration which inhibits the 50% of cell survival). The plates were incubated at 37° C. for 72 h. At the end of the treatment, the plates were washed by removal of the supernatant and addition of PBS 3 times. 200 μl PBS and 50 μl of cold 80% trichloroacetic acid (TCA) were added. The plates were incubated on ice for at least 1 h. TCA was removed and the plates were washed 3 times by immersion in distilled-water and dried on paper and at 40° C. for 5 min. Then 200 μl of 0.4% sulphorodamine B in 1% acetic acid were added. The plates were incubated at RT for further 30 min. Sulphorodamine B was removed, the plates were washed by immersion in 1% acetic acid 3 times, then they were dried on paper and at 40° C. for 5 min. Then 200 μl Tris 10 mM were added, the plates were kept under stirring for 20 min. The cell survival was determined by means of optical density using a Multiskan spectrofluorimeter at 540 nm. The amount of cells killed was calculated as the percentage decrease in sulphorodamine B binding compared with control cultures.

The IC₅₀ values were calculated with the “ALLFIT” program.

The antiproliferative activity of the three conjugates was compared on two human tumour cell lines (A2780 ovarian tumour cells with high levels of integrin and PC3 prostate tumour cells with low levels of integrin). The molecules showed a marked cytotoxic potency on tumour cells with IC₅₀ values 8 nM as shown in table 4. All the conjugates revealed a minor effect on PC3 tumour cells with low levels of integrin (IC₅₀ values ranged from 1 to 4.6 μM). In particular, three compounds presented a rather specific antiproliferative effect on A2780 tumour cells with respect to that observed on PC3 tumour cells (table 4) with a potency roughly hundred fold greater on the former.

TABLE 4
Cytotoxicity of the conjugates on A2780 ovarian carcinoma cells and PC3
prostate carcinoma cells (72 h of treatment)

| | PC3 | A2780 |
|---|---|---|
| Compound | IC50 ± SD, μM | |
| ST3280 | 0.2 ± 0.03 | 0.0084 ± 0.0006 |
| ST3833 | 4.6 ± 0.8 | 0.095 ± 0.02 |
| ST4167 | 1.0 ± 0.1 | 0.030 ± 0.003 |
| ST4215 | 2.5 ± 0.7 | 0.009 ± 0.0007 |
| ST5744TF1 | 1.0 ± 0.02 | 0.008 ± 0.0005 |
| ST5745TF1 | 1.0 ± 0.01 | 0.008 ± 0.0001 |

### In Vivo Evaluation of Antitumour Activity of the Conjugate ST3833 on Tumour Growth of Ovarian Carcinoma Xenografted in CD1 Nude Mice

Tumour cell lines (3×10⁶) were injected s.c. into the right flank of CD1 nude mice (Harlan). Each experimental group included 10 mice. Tumours were implanted on day 0, and tumour growth was followed by biweekly measurements of tumour diameters with a Vernier caliper. Tumour volume was calculated according to the formula: TV (mm³)=d²×D/2, where d and D are the shortest diameter and the longest diameter respectively. Drug treatment started when tumours were just measurable on day 3 after tumour inoculation. The drug was administered subcutaneously for two weeks according to the schedule qd×5/w×2w at different doses in a volume of 10 ml/kg. Control mice were treated with the vehicle (10% DMSO).

Drug efficacy was assessed as described below.
a) TVI in drug-treated versus control mice was expressed as follows: TVI (%)=100−(mean TV treated/mean TV control)×100. TVI was evaluated 6 days after the last treatment, this timing corresponding to the time necessary to observe a doubling of tumour volume in the control mice.
b) Log cell kill (LCK) was calculated using the following formula: LCK=(T−C)/3.32×DT where T and C are the mean time (in days) required for treated (T) and control (C) tumours, respectively to reach a determined volume, and DT is the time necessary to observe a doubling of tumour volume in the control mice.
c) CR was defined as disappearance of the tumour lasting at least 6 days after the end of treatments. Tumours that had not regrown by the end of the experiment were considered “cured”.

Toxic effects of drug treatment were assessed as described below.
a) BWL was calculated as follows: BWL (%)=100−(mean body weight day x/mean body weight day 1)×100, where day 1 is the first day of treatment, and day x is any day thereafter. The highest (maximum) BWL is reported in the table. Mice were weighed every day throughout the period of treatment.
b) Lethal toxicity was defined as any death in treated groups occurring before any control death. Mice were inspected daily for mortality.

TI (therapeutic index) was calculated as ratio MTD/ED80.

### Results

The antitumour activity of ST3833 was investigated against the tumour most responsive in vitro xenografted in CD1 nude mice. The molecule showed an approximate maximum tolerate dose (MTD) of 25 mg/kg delivered s.c. according to the schedule qd×5/w×2w since BWL was 25% and 1 out 10 mice died. ST3833 revealed a potent antitumour effect since it produced a complete regression of all tumours (cured mice at day 90 were 100% at the MTD) (table 5). At ⅓ MTD (8.3 mg/kg) 50% cured mice were observed. At lower doses (2.77 and 0.92 mg/kg), cured mice were 30%. The persistence of effect after the last treatment and the good tolerability of the conjugate showed a high therapeutic index (TI=8.9), suggesting a high therapeutic potential for the conjugate.

TABLE 5
Antitumour activity of ST3833 delivered subcutanously (qdx5/wx2w)
against A2780 ovarian ca. xenografted in CD1 nude mice

| | ^{a}Dose | | ^{c}Lethal | | | | | |
|---|---|---|---|---|---|---|---|---|
| Compound | mg/10 ml/kg | ^{b}BWL % | toxicity | ^{d}TVI % | ^{e}CR | ^{f}Cured | ^{g}LCK | ^{h}TI |
| ST3833 | 25 | 24 | 1/10 | 100 | 9/9 | 9/9 | >11.4 | 8.9 |
| | 8.3 | 0 | 0/10 | 100 | 5/10 | 4/10 | 2.7 | |
| | 2.77 | 0 | 0/10 | 75 | 3/10 | 3/10 | 1.1 | |
| | 0.92 | 0 | 0/10 | 60 | 3/10 | 3/10 | 0.9 | |

^{a}Subcutanous dose used in each administration.
^{b}Maximum BWL percentage due to the drug treatment.
^{c}Dead/treated animals.
^{d}TVI percentage versus control mice.
^{e}CR: disappearance of tumour for at least 10 days.
^{f}Cured: mice without lesion 90 days after tumour injection.
^{g}LCK, see Methods.
^{h}TI: therapeutic index (MTD/ED80).

### In Vivo Evaluation of Antimetastatic Activity of the Conjugate ST3833 on Bone Metastases Induced by Intracardiac Injection of PC3 Human Prostate Carcinoma

Male CD1 nude mice were anesthetized by 4 ml/kg of a mixture (xylazine:ketavet 100) given i.p. PC3 tumour cells were inoculated by intracardiac injection (1×10⁵ cells/0.1 ml/mouse) into the heart left ventricle of mice using a 27-gauge needle. Mice were subdivided (11 mice/group) in the following experimental groups and after three days from tumour injection the molecules were administered as described:

Vehicle (DMSO 10%) i.v. q4d×4.

ST3833 56 mg/10 ml/kg i.v. q4d×4

To evaluate the antitumour activity of the drug, high-resolution total body radiological analysis was carried out by using Faxitron system. Radiological analysis was carried out 30 days after tumour injection. Body weight recordings were carried out through the study and mortality was noted.

The conjugate showed to be well tolerated at 56 mg/kg iv (q4d×4) since no reduction of body weight of lethal toxicity was found. The molecule revealed to significantly increase the life of span of 45% (P<0.001) and to reduce the incidence of osteolytic lesions from 91% of mice in vehicle-treated group to 45% of mice in drug-treated group (table 6).

TABLE 6
Antimetastatic activity of ST3833 delivered intravenously (q4dx4)
against PC3 prostate ca. xenografted in CD1 nude mice

| | ^{a}Dose mg/ | | | ^{d}Incidence | ^{e}MST | |
|---|---|---|---|---|---|---|
| | 10 ml/kg | | ^{c}Lethal | of osteolytic | (range | |
| Compound | se | ^{b}BWL % | toxicity | lesions | days) | ^{f}ILS % |
| Vehicle | 0 | 0 | 0/11 | 10/11 | 40 | / |
| | | | | | (37-45) | |
| ST3833 | 56 | 0 | 0/11 | 5/11 | 58 | ***45 |
| | | | | | (45-71) | |

^{a}Intravenous dose used in each administration.
^{b}Maximum BWL percentage due to the drug treatment.
^{c}Dead/treated animals.
^{d}Incidence of osteolytic lesions (number of drug-treated mice with metastases vs vehicle-treated mice 30 days after tumour injection.
^{e}MST: median survival of time.
^{f}ILS %: increase in life span.
***P < 0.001 vs vehicle-treated group (Mann-Whitney test).

1. A cyclic peptide of formula I
[(L-D)nE]ₘ-F-D-PI-SI-CT Formula I
wherein,
L is a recognizing α-integrin receptor cyclic peptide of formula II
c(Rⁱ-Arg-Gly-Asp-R²) Formula II
R¹ is Amp, Lys or Aad;
R² is Phe, Tyr or Amp with the R-configuration;
D at each occurrence is the same or different, is absent or is a divalent group of formula III
SPⁱ-Aⁱ-SP²-A²-SP³ Formula III
wherein SP¹ is absent or is R³—(CH₂)_{q}—(OCH2—CH2)_{q}—O—(CH₂)_{q}—R⁴;
R³ and R⁴, are the same or different, are absent, or —CO—, —COO—, —NH—, —O—, or a divalent radical of formula IV, formula VIII or formula IX 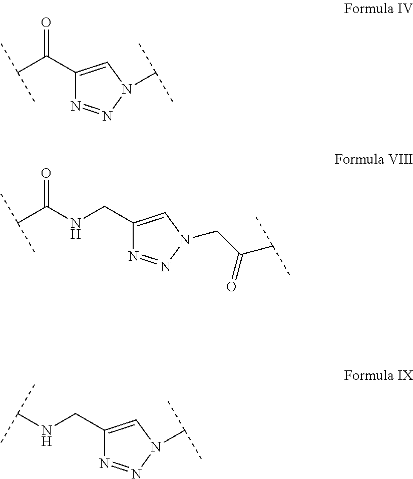
q at each occurrence are the same or different and are independently an integer comprised between 0-6;
A¹ is absent or a natural or unnatural, (L) or (D)-amino acid bearing a hydrophilic side chain;
SP² is absent or the same as SP¹;
A² is absent or the same as A¹;
SP³ is absent or the same as SP¹;
m=1 or 2;
n=1 or 2;
E at each occurrence can be the same or different and is GIu, Lys or is absent; F is the same as E or is absent or is a histidine analogue of formula X; 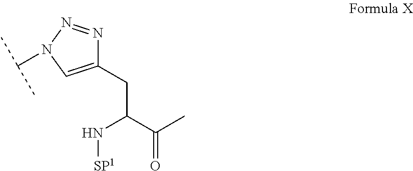
wherein the triazole ring is linked to the D-PI-SI-CT moiety, the carbonyl moiety is linked to the L-containing moiety and SP¹ is as defined above;
PI is a natural or unnatural oligopeptide, made of (L) or (D) amino acids selected between Ala and Cit;
SI is the divalent radical p-aminobenzyloxycarbonyl;
CT represents a cytotoxic radical;
their tautomers, their geometrical isomers, their optically active forms such as enantiomers, diastereomers and their racemate forms, as well as their pharmaceutically acceptable salts thereof;
with the following proviso:
at least one D should be present;
and when E is present, it is linked to the portion bearing the L group through its amino moieties when E is Lys, or through its carboxyl moieties when E is GIu.

2. A cyclic peptide according to claim 1 wherein CT is a camptothecin derivative, R¹ is Amp or Aad, R² is chosen from Phe, Amp or Tyr.

3. A cyclic peptide according to claim 1 wherein m=1 and n=1.

4. A cyclic peptide according to claim 1 wherein m=1 and n=2.

5. A medicament comprising cyclic peptides according to claim 1 endowed with integrin αᵥβ₃ and αᵥβδ inhibitory properties.

6. The medicament according to claim 5, having an integrin IC50 less than 1 μM.

7. Pharmaceutical compositions containing at least one cyclic peptide according to claim 1 in a mixture with at least one pharmaceutically acceptable excipient and/or vehicle.

8. Process for synthesizing cyclic peptides according to claim 1, said method comprising reacting compounds of formula V
(CT-SI-PI)-NH₂ (formula V)
wherein CT, SI and PI are as described above,
with an azide containing derivative of formula VI
L-(SP¹-A¹-SP²-A²-SP³)-N₃ (formula VI)
wherein L, SP¹, A¹, SP², A² and SP³ are as described above with R⁴ being CO wherein CT, SI and PI are as described above.

9. Process for synthesizing cyclic peptides according to claim 1, said method comprising reacting compounds of formula VII
(CT-SI-PI)-CO—C≡CH (formula VII)
wherein CT, SI and PI are as described above,
with compounds of formula VI,
wherein L, SP¹, A¹, SP², A² and SP³ in the compounds of Formula VI are as described above with the proviso that R⁴ is absent.

10. Process for synthesizing cyclic peptides according to claim 1, said method comprising reacting compounds of formula XI
(CT-SI-PI)-D-NHCH₂—C≡CH (formula XI)
wherein CT, SI, PI and D are as described above,
with compounds of formula XII
[(L-D)ₙE]₁ₙ-COCH₂—N₃ (formula XII)
wherein L, D and E are as described above.

11. Process for synthesizing cyclic peptides according to claim 1, said method comprising reacting compounds of formula XIII
(CT-SI-PI)-D-N₃ (formula XIII)
wherein CT, SI, PI and D are as described above,
with compounds of formula XIV
[(L-D)ₙE]ₘ-CO—CH(NHD)CH₂—C≡CH (formula XIV)
wherein L, D and E are as described above.

12. (canceled)

13. A method of treating a mammal suffering from an uncontrolled cellular growth, invasion and/or metastasis condition, said method comprising administering a therapeutically effective amount of a pharmaceutical composition according to claim 3 to a mammal suffering from uncontrolled cellular growth; and treating said mammal.

14. A method according to claim 13, wherein said uncontrolled cellular growth comprises ovarian and/or prostate carcinoma.

15. A method according to claim 13, wherein said therapeutically effective amount is from 0.01 mg/kg to 100 mg/kg.

16. A method according to claim 13, wherein said therapeutically effective amount is from 0.05 mg/kg to 50 mg/kg.
